(19) United States
(12) Patent Application Publication
(10) Pub. No.: US 2010/0210643 A1
(43) Pub. Date: Aug. 19, 2010

(54) sGC STIMULATORS, sGC ACTIVATORS AND COMBINATIONS FOR THE TREATMENT OF UROLOGICAL DISORDERS

(75) Inventors: Peter Sandner, Wuppertal (DE); Hanna Tinel, Wuppertal (DE); Johannes-Peter Stasch, Solingen (DE); Dieter Neuser, Langenfeld (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/599,695

(22) PCT Filed: Apr. 29, 2008
(86) PCT No.: PCT/EP2008/003450

§ 371 Date: Nov. 11, 2009

(30) Foreign Application Priority Data
May 12, 2007 (EP) 07009557.5

(51) Int. Cl. A61K 31/5377; A61K 31/505; A61K 31/216; A61K 31/53; A61K 31/496; A61K 31/4985; A61K 31/519; A61K 31/506; C07D 403/14; C07C 229/38; A61P 13/00; A61P 13/10; A61P 13/02

(52) U.S. Cl. 514/234.2; 514/256; 514/533; 514/243; 514/252.16; 514/250; 514/262.1; 514/275; 544/333; 560/45

(57) ABSTRACT

The invention provides pharmacological compositions comprising a stimulator or activator of the soluble guanylate cyclase alone (sGC) or in combination with a PDE5 inhibitor for the treatment of urological disorders comprising of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI) and pelvic pain (PP).

### TECHNICAL FIELD OF THE INVENTION

The present invention relates to soluble guanylate cyclase (sGC) and to phosphodiesterases (PDEs) and the pharmacology of sGC stimulators, sGC activators and PDE inhibitors. More particularly, the invention relates to the use of sGC stimulators and sGC activators alone and in combination with PDE5 inhibitors for preparation of medicaments for the treatment of urological disorders.

### BACKGROUND OF THE INVENTION

Benign prostate syndrome (BPS) comprises lower urinary tract symptoms (LUTS) and benign prostate hyperplasia (BPH), which can cause bladder outlet obstruction (BOO). LUTS may also be present in patients without benign prostate enlargement (BPE). LUTS comprises obstructive symptoms and irritative symptoms: obstructive symptoms are induced by compression of the urethra caused by BPE; irritative symptoms occur during the filling phase of the bladder and are characterized by non-voiding contractions of the bladder detrusor muscle (Abrahms 1994, Holtgreve 1998, Chapple 2006). LUTS considerably decreases the quality of life of affected patients and is a major healthcare problem in developed countries. Besides prostatic surgery (20% of all BPH patients), the common treatment of the disease comprises 5-alpha reductase inhibitors (finasteride) and alpha blockers (tamsulosin, doxazosin, terazosin, alfuzosin) (Truss 2001). 5-alpha reductase inhibitors influence the mechanical component of BPH and inhibit proliferation of the prostate tissue. Alpha blockers influence the dynamic component and decrease the irritative symptoms of BPH via relaxation of the prostatic smooth muscle which decreases the urethral resistance. Moreover alpha-blockers are able to relax directly bladder smooth muscle cells and reduce the non voiding contractions of the bladder. Overactive bladder (OAB) is a term, defined by the International Continence Society and is usually described as urgency with or without incontinence, frequency and nocturia (Abrahms 2002). Urgency urinary incontinence (UUI) is a sudden and compelling desire to pass urine which can be accompanied by the involuntary leakage of urine. In addition, OAB patients display higher voiding frequency, with >8 episodes of voiding during day-time, and nocturia, with more then 1 voiding during sleeping-time. Urge urinary incontinence (UUI) is the most bothersome symptom of OAB and is associated with a significantly reduced quality of life of the patients (Milsom 2001). The pathophysiology of these diseases is not fully understood and seemed to be multi-factorial. The voiding process is normally tightly regulated by neural circuits within the brain and the spinal cord, which co-ordinate smooth muscle activity in bladder and urethra. Direct contraction and relaxation of the bladder smooth muscle plays a critical role in this disease (Andersson 2004). In addition to local application of botulinum toxin or resiniferatoxin (Sahai 2006, Patterson 2006) the main treatment option is based on muscarinic receptor antagonists (antimuscarinics). These prevent acetylcholine-induced contraction of the detrusor muscle by blocking post-synaptic M3 receptors during the filling phase (Sahai 2006, Chapple 2006). There is evidence that LUTS and OAB comprising the same symptoms, are caused by similar pathomechanism and arise from a dysfunctional bladder. Since the treatment options for BPS, LUTS, OAB, HUT have limited efficacy and/or unvavorable side effect profile, the development of new treatment options might improve the quality of life in patients significantly.

It is well established that the cyclic nucleotides cAMP and cGMP can reduce smooth muscle tone (Drescher 1994). cAMP and cGMP are synthesized from their corresponding nucleoside triphosphates by the adenylate and guanylate cyclase respectively. They are degraded by the cyclic nucleotide phosphodiesterases (PDEs) which regulate the intracellular cAMP and cGMP level very effectively. Up to now 11 different PDE family members have been identified which differ in structure, regulation and specificity for their substrate (Soderling 2000). The role of PDEs for the treatment of Urological Disorders is only poorly understood, the characterization of PDE isoforms has lagged behind other systems and much of the literature was published prior to identification of the newly identified PDEs. Although PDEs are expressed in the lower urinary tract i.e. in bladder, urethral and in prostate tissues, mRNA expression data and direct comparisons of all PDE isogenes are still missing or inconsistent. There are some evidences that unspecific PDE inhibition is able to relax human prostate tissue (Drescher 1994). The data about the effect of PDE-5 inhibition is very limited. It has been shown, that Zaprinast, a PDE-5 inhibitor which also inhibits PDE-6, -9 and -11 is able to relax pre-contracted human prostate tissue in vitro (Uckert 2001), whereas the role of other PDE families within this tissue needs to be determined. Within the bladder, unspecific blockade of different PDEs by IBMX (inhibition of PDE-1, -2, -3, -4, -5, -6, -10, and -11) could relax bladder of female Guinea Pigs whereas Zaprinast was ineffective (Gillespie 2004). The role of cGMP elevation with NO donors on bladder function is even less clear. It was shown in guinea pigs that urothelial, suburothelial and intramural neurons contain considerable amounts of endothelial NO synthase (eNOS) and NO-inducible cGMP, which may be important for bladder sensations and the micturition threshold (Gillespie 2004, 2006) which might imply a role for the NO/cGMP system.

Soluble guanylate cyclase (sGC) is a key signal-transduction enzyme that is activated by nitric oxide (NO). Impaired bioavailability and/or responsiveness to endogenous NO have been implicated in the pathogenesis of cardiovascular, endothelial, renal, hepatic, sexual and urological dysfunctions. Correspondingly, nitrates and various ‘NO-donor’ drugs have been used for treating some of these conditions. However, these therapies have important limitations including non-specific interactions of NO with other biomolecules. Compounds that activate sGC in a NO-independent manner might therefore offer a considerable advantage over current therapies. Two classes of compounds have been identified recently that activate the sGC NO-independently, the heme-dependent sGC stimulators, such as BAY 41-2272, BAY 41-8543 and BAY 63-2521 and heme-independent sGC activators, such as BAY 58-2667 and HMR-1766 (for review see Evgenov et al., 2006).

### DISCLOSURE OF THE INVENTION

Urological disorders addressed by therapeutic agents of the invention comprise Benign Prostate Syndrome (BSE), Benign Prostate Hyperplasia (BPH), Benign Prostate Enlargement (BPE) Lower Urinary Tract Symptoms (LUTS) and in particular irritative symptoms caused by BPH-induced bladder outlet obstruction (BOO). Because not only symptomatic irritations of the bladder but also underlying BPE-induced bladder outlet obstructions are addressed by treatment with specific sGC stimulators and sGC activators alone or in combination with PDE5 inhibitors this treatment provides substantial advantage over methods of treatment already known in the art.

Moreover urological disorders addressed by therapeutic agents of the invention which in particular and with substantial advantage can be treated by the above mentioned sGC stimulators and sGC activators alone, or in combination with PDE5 inhibitors, are genitourinary disorders comprising neurogenic bladder syndrome [also referred to as overactive bladder (OAB) or interstitial cystitis (IC)], urinary incontinence (UI) like mixed-, urge-, stress-, or overflow incontinence (MUI, UUI, SUI, OUI), pelvic pain (PP), benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies and erectile dysfunction.

The invention provides sGC stimulators and sGC activators alone or in combination with PDE5 inhibitors which are useful for the treatment of urological disorders especially BPS, BPH, BPE, LUTS, OAB, UI, UUI, MUI, SUI, OUI, IC, PP.

Guanylate cyclase (sGC) stimulator and sGC activator is preferably a compound selected from the group consisting of
2-[1-(2-fluorobenzyl)-1H-pyrazolo[3,4-b]pyridin-3-yl]-5-(4-morpholinyl)-4,6-pyrimidine-diamine (1), described also as example 16 in WO 00/06569, herein incorporated by reference,
2-[1-(2-fluorobenzyl)-1H-pyrazolo[3,4-b]pyridin-3-yl]-5-(4-pyridinyl)-4-pyrimidinamine (2), described also as example 1 in WO 02/42301, herein incorporated by reference,
methyl-4,6-diamino-2-[1-(2-fluorobenzyl)-1H-pyrazolo[3,4-b]pyridin-3-yl]-5-pyrimidinyl-(methyl)carbamate (3), described also as example 8 in WO 03/095451, herein incorporated by reference,
methyl-4,6-diamino-2-[1-(2-fluorobenzyl)-1H-pyrazolo[3,4-b]pyridin-3-yl]-5-pyrimidinyl-carbamate (4), described also as example 5 in WO 03/095451, herein incorporated by reference.
   and
4-({(4-carboxybutyl)[2-(2-{[4-(2-phenylethyl)benzyl]oxy}phenyl)ethyl]amino}methyl)benzoic acid (5)

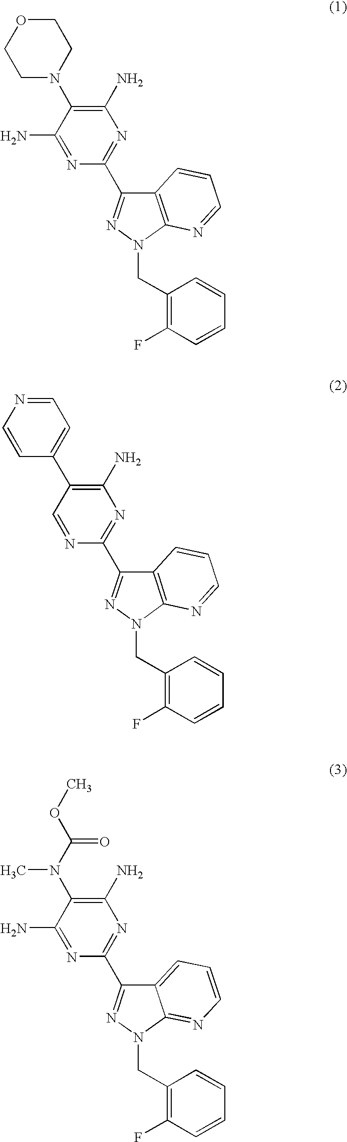

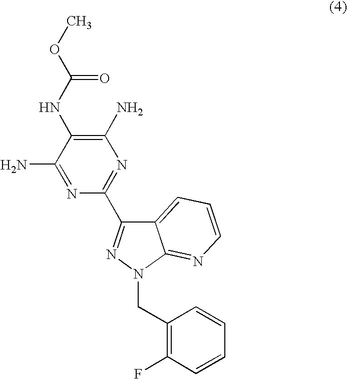

Compounds (1), (2), (3) and (4) are known soluble guanylate cyclase (sGC) stimulators which have been previously described for the treatment of stable angina pectoris or erectile dysfunction.

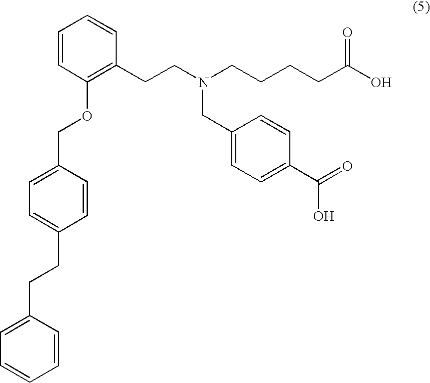

Compound (5) is known as sGC activator

PDE-5 inhibitors which are useful for the combined treatment of urological disorders are in particular Tadalafil ((6R,12aR)-2,3,6,7,12,12a-Hexahydro-2-methyl-6-(3,4-methylenedioxyphenyl)pyrazino(1′,2′:1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (1,2,4)triazin-4-one), Sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), Udenafil 5-[2-propyloxy-5-(1-methyl-2-pyrrolidinylethylamidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxyphenyl)methyl]amino}-2-[(2S)-2-(hydroxymethyl)pyrrolidin-1-yl]-N-(pyrimidin-2-ylmethyl)pyrimidine-5-carboxamide, SLx 2101 of Surface Logix, LAS 34179Triazolo[1,2-]xanthine, 6-methyl-4-propyl-2-[2-propoxy-5-(4-methylpiperazino)sulfonyl]phenyl-, or salts, hydrates or hydrates of salts of the before mentioned PDE5 inhibitors

A pharmaceutical composition of the invention is formulated to be compatible with its intended route of administration. Examples of routes of administration include parenteral e.g., intravenous, intradermal, subcutaneous' oral (e.g. ‘inhalation)’ transdermal (topical) transmucosal and rectal administration. Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents for example, parabens, chlorobutanol, phenol, ascorbic acid, thimerosal, and the like. In many cases, it will be preferable to include isotonic agents, for example, sugars, polyalcohols such as maitol sorbitol sodium chloride in the composition.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or con1 starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g.′ a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Bio degradable, biocompatible polymers can be used, such as ethylene vinyl acetate, polyanhydrides, polyglycolic acid, collagen, polyorthoesters, and polylactic acid.

In another embodiment the invention provides sGC stimulators and sGC activators in combination with PDE5 inhibitors and their use for the preparation of pharmaceutical compositions for the treatment of urological disorders, whereby these combinations comprise either i) pharmaceutical compositions comprising a compound having a sGC stimulatory or activatory action and PDE-5 inhibitory activity, or ii) pharmaceutical compositions comprising one sGC stimulator and sGC activator and at least one PDE-5 inhibitor as a fixed combination in one application unit, or iii) a kit of parts containing at least two sets of pharmaceutical compositions, each set consisting of at least one pharmaceutical preparation comprising a PDE-5 inhibitor in units of at least one dose and at least one pharmaceutical preparation comprising a sGC activator or sGC stimulator in units of at least one dose, whereby each application unit of said pharmaceutical compositions is administered in combination, sequentially, as single dose or in multiple doses.

In particular, the present invention provides:

A pharmaceutical composition for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal, comprising a therapeutic agent which regulates the activity of the soluble guanylate cyclase.

A pharmaceutical composition for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal comprising a therapeutic agent which is a stimulator or activator of the soluble guanylate cyclase from the group of sGC activators and stimulators consisting of
2-[1-(2-fluorobenzyl)-1H-pyrazolo[3,4-b]pyridin-3-yl]-5-(4-morpholinyl)-4,6-pyrimidinediamine (1),
2-[1-(2-fluorobenzyl)-1H-pyrazolo[3,4-b]pyridin-3-yl]-5-(4-pyridinyl)-4-pyrimidinamine (2),
methyl-4,6-diamino-2-[1-(2-fluorobenzyl)-1H-pyrazolo[3,4-b]pyridin-3-yl]-5-pyrimidinyl-(methyl)carbamate (3),
methyl-4,6-diamino-2-[1-(2-fluorobenzyl)-1H-pyrazolo[3,4-b]pyridin-3-yl]-5-pyrimidinyl-carbamate (4).
and
4-({(4-carboxybutyl)[2-(2-{[4-(2-phenylethyl)benzyl]oxy}phenyl)ethyl]amino}methyl)benzoic acid (5)

A pharmaceutical composition for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal, containing at least one compound selected from
2-[1-(2-fluorobenzyl)-1H-pyrazolo[3,4-b]pyridin-3-yl]-5-(4-morpholinyl)-4,6-pyrimidinediamine (1),
2-[1-(2-fluorobenzyl)-1H-pyrazolo[3,4-b]pyridin-3-yl]-5-(4-pyridinyl)-4-pyrimidinamine (2),
methyl-4,6-diamino-2-[1-(2-fluorobenzyl)-1H-pyrazolo[3,4-b]pyridin-3-yl]-5-pyrimidinyl-(methyl)carbamate (3), and
methyl-4,6-diamino-2-[1-(2-fluorobenzyl)-1H-pyrazolo[3,4-b]pyridin-3-yl]-5-pyrimidinyl-carbamate (4),
and
4-({(4-carboxybutyl)[2-(2-{[4-(2-phenylethyl)benzyl]oxy}phenyl)ethyl]amino}methyl)benzoic acid (5)
   and at least one compound selected from
   Tadalafil ((6R,12aR)-2,3,6,7,12,12a-Hexahydro-2-methyl-6-(3,4-methylene-dioxyphenyl)pyrazino(1′,2′:1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Sildenafil (3-[2-ethoxy-5-(4-methylpiperazin-1-yl)sulfonyl-phenyl]-7-methy-l-9-propyl-2,4,7,8-tetrazabicyclo[4.3.0]nona-3,8,10-trien-5-one), Udenafil 5-[2-propyloxy-5-(1-methyl-2-pyrrolidinyl-ethyl-amido sulfonyl)phenyl]-methyl-3-propyl-1,6-dihydro-7H-pyrazol o(4,3-d)pyrimidine-7-one,
   Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxy phenyl)methyl]amino}-2-[(2S)-2-(hydroxymethyl)pyrrolidin-1-yl]-N-(pyrimidin-2-yl methyl)pyrimidine-5-carboxamide, SLx 2101 of Surface Logix, and/or LAS 34179Triazolo[1,2-]xanthine, 6-methyl-4-propyl-2-[2-propoxy-5-(4-methylpiperazino)-sulfonyl]phenyl or salts, hydrates or hydrates of salts of the before mentioned PDE5 inhibitors.

Use of a sGC stimulator and activator for the preparation of a pharmaceutical composition for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal.

Use of a combination of at least one sGC stimulator or activator and at least one PDE5 inhibitor for the preparation of a pharmaceutical composition for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal.

Use of sGC stimulator or activator selected from the group of sGC stimulators and activators of 2-[1-(2-fluorobenzyl)-1H-pyrazolo[3,4-b]pyridin-3-yl]-5-(4-morpholinyl)-4,6-pyrimidinediamine(1), 2-[1-(2-fluorobenzyl)-1H-pyrazolo[3,4-b]pyridin-3-yl]-5-(4-pyridinyl)-4-pyrimidinamine (2), methyl-4,6-diamino-2-[1-(2-fluorobenzyl)-1H-pyrazolo[3,4-b]pyridin-3-yl]-5-pyrimidinyl-(methyl)carbamate (3), methyl-4,6-diamino-2-[1-(2-fluorobenzyl)-1H-pyrazolo[3,4-b]pyridin-3-yl]-5-pyrimidinylcarbamate (4), and 4-({(4-carboxybutyl)[2-(2-{[4-(2-phenylethyl)benzyl]oxy}phenyl)ethyl]amino}methyl)benzoic acid (5) for the preparation of a pharmaceutical composition for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal.

Use of a combination of at least one sGC stimulator and activator selected from the group of sGC stimulators and activators of 2-[1-(2-fluorobenzyl)-1H-pyrazolo[3,4-b]pyridin-3-yl]-5-(4-morpholinyl)-4,6-pyrimidinediamine (1), 2-[1-(2-fluorobenzyl)-1H-pyrazolo[3,4-b]pyridin-3-yl]-5-(4-pyridinyl)-4-pyrimidinamine (2), methyl-4,6-diamino-2-[1-(2-fluorobenzyl)-1H-pyrazolo[3,4-b]pyridin-3-yl]-5-pyrimidinyl(methyl)carbamate (3),
methyl-4,6-diamino-2-[1-(2-fluorobenzyl)-1H-pyrazolo[3,4-b]pyridin-3-yl]-5-pyrimidinyl-carbamate (4), and 4-({(4-carboxybutyl)[2-(2-{[4-(2-phenylethyl)benzyl]oxy}phenyl)ethyl]amino}methyl)benzoic acid (5)
   and at least one PDE-5 inhibitor selected from the group of PDE-5 inhibitors consisting of Vardenafil (2-(2-Ethoxy-5-(4-ethylpiperazin-1-yl-1-sulfonyl)phenyl)-5-methyl-7-propyl-3H-imidazo (5,1-f) (1,2,4) triazin-4-one), Sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), and Tadalafil ((6R,12aR)-2,3,6,7,12,12a-Hexahydro-2-methyl-6-(3,4-methylene-dioxyphenyl) for the preparation of a pharmaceutical composition for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal.

A method for the preparation of a pharmaceutical composition for the treatment of the diseases as mentioned above wherein stimulator and activator of the soluble guanylate-cyclase is selected from the group of compounds consisting of 2-[1-(2-fluorobenzyl)-1H-pyrazolo[3,4-b]pyridin-3-yl]-5-(4-morpholinyl)-4,6-pyrimidinediamine (1),
2-[1-(2-fluorobenzyl)-1H-pyrazolo[3,4-b]pyridin-3-yl]-5-(4-pyridinyl)-4-pyrimidinamine (2), methyl-4,6-diamino-2-[1-(2-fluorobenzyl)-1H-pyrazolo[3,4-b]pyridin-3-yl]-5-pyrimidinyl-(methyl)carbamate (3), methyl-4,6-diamino-2-[1-(2-fluorobenzyl)-1H-pyrazolo[3,4-b]pyridin-3-yl]-5-pyrimidinylcarbamate (4),
and
4-({(4-carboxybutyl)[2-(2-{[4-(2-phenylethyl)benzyl]oxy}phenyl)ethyl]amino}methyl)benzoic acid (5).

Use of a pharmaceutical composition as mentioned above for the stimulation and activation of the soluble guanylate cyclase in a mammal having a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UM, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies.

Use of a pharmaceutical composition as mentioned above for the stimulation and activation of the soluble guanylate cyclase and for the regulation of PDE activity in a mammal having a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies.

A kit of parts for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal including humans containing a combination of at least one pharmaceutical composition selected from the group of pharmaceutical compositions consisting of Vardenafil, Sildenafil and Tadalafil and at least one pharmaceutical composition selected from the group of sGC stimulators and activators.

In particular, the present invention provides:

A pharmaceutical composition containing methyl-4,6-diamino-2-[1-(2-fluorobenzyl)-1H-pyrazolo[3,4-b]pyridin-3-yl]-5-pyrimidinyl carbamate (4) and/or 4-({(4-carboxybutyl)[2-(2-{[4-(2-phenylethyl)benzyl]oxy}phenyl)ethyl]amino}methyl)benzoic acid (5), for the treatment of neurogenic bladder, overactive bladder and interstitial cystitis.

A pharmaceutical composition containing at least methyl-4,6-diamino-2-[1-(2-fluorobenzyl)-1H-pyrazolo[3,4-b]pyridin-3-yl]-5-pyrimidinylcarbamate (4) and/or 4-({(4-carboxybutyl)[2-(2-{[4-(2-phenylethyl)benzyl]oxy}phenyl)ethyl]amino}methyl)benzoic acid (5) and at least Sildenafil, Tadalafil, Udenafil, Dasantafil, Avanafil, SLx2101 and LAS34179 and preferably Vardenafil or a salt, a hydrat or a hydrat of a salt of the before mentioned PDE5 inhibitors, for the treatment of neurogenic bladder, overactive bladder and interstitial cystitis.

In order to clarify the effect of sGC stimulators and sGC activators alone and in combination with vardenafil experiments are performed. In particular sGC and PDE5 in organs of the lower urinary tract are semiquantitated, the functional activity of sGC stimulators, sGC activators and PDE5 inhibitors in vitro on tissues of the lower urinary tract of different mammals are assessed, the effect of sGC stimulators, sGC activators and PDE5 inhibitors on prostate cells are assessed, the functional activity of sGC stimulators and activators and PDE5 inhibitors in vivo are assessed in models for urological diseases, especially on voiding parameters in i.e. non-obstructed, obstructed, incontinent, cystitic, animals.

### REFERENCE

Abrams P, Cardozo L, Fall M, Griffiths D, Rosier P, Ulmsten U, et al. The standardisation of terminology of lower urinary tract function: Report from the Standardisation Sub-committee of the International Continence Society. Neurourol Urodyn 2002; 21: 167-178.
Abrams P. New words for old: lower urinary tract symptoms for prostatism. BMJ 1994; 308: 929-930.
Andersson K E, Arner A. Urinary Bladder Contraction and Relaxation: Physiology and Pathophysiology. Physiol. Rev 2004; 84: 935-986.
Andersson K E, Chapple C R, Hofner K. Future drugs for the treatment of benign prostatic hyperplasia. World J Urol 2002; 19:436-42.
Bardelle, C. et al. (1999) Anal. Biochem. 275: 148-155
Carbone D J Jr, Hodges S: Medical therapy for benign prostatic hyperplasia: sexual dysfunction and impact on quality of life. Int J Impot Res 2003; 15:299-306.
Chapple C R, Roehrborn C G. A shifted paradigm for the further understanding, evaluation, and treatment of lower urinary tract symptoms in men: focus on the bladder. European Urology 2006; 49: 651-659.
Desgrandchamps F. Clinical relevance of growth factor antagonists in the treatment of benign prostatic hyperplasia. Eur Urol. 1997; 32:28-31.
Drescher P, Eckert R E, Madsen P O. Smooth muscle contractility in prostatic hyperplasia: role of cyclic adenosine monophosphate. Prostate 1994; 25:76.
Evgenov O V, Pacher P, Schmidt P M, Hasidó G, Schmidt H H H W, Stasch J P. NO-independent stimulators and activators of soluble guanylate cyclase: discovery and therapeutic potential. Nature Rev—Drug Disc. 2006; 5: 755-768.
Gillespie J I, Drake M J. Phosphodiesterase-linked inhibition of nonmicturition activity in the isolated bladder. BJU International 2004; 93:1325-1332
Gillespie J I, Markerink-van Ittersum M, De Vente J. Endogenous nitric oxide/cGMP signaling in the guinea pig bladder: evidence for distinct population of sub-urothelial interstitial cells. Cell Tissue Res 2006; 325: 325-332.
Gopalakrishnan M, Shieh C C. Potassium channel subtypes as molecular targets for overactive bladder and other urological disorders. Expert Opin Ther Targets 2004; 8:437-58.
Guess H A, Epidemiology and natural history of benign prostatic hyperplasia. Urol Clin North Am 1995; 22:247.
Hansen, R. S., and Beavo, J. A., PITAS USA 1982, 79: 2788-92
Heid C A, Stevens J, Livak K J, Williams P M, Real time quantitative PCR. Genome Res 1996; 6:986-994.
Holtgrewe H L. Current trend in management of men with lower urinary tract symptoms and benign prostate hyperplasia. Urology 1998; 51: 1-7.
Milsom I, Abrams P, Cardozo L, Roberts R G, Thüroff J, Wein A J. How widespread are the symptoms of the overactive bladder and how are they managed? A population based prevalence study. BJU Int 2001; 87: 760-766.
Patterson J M, Chapple C R. Botulinum toxin in urinary incontinence. Curr Opin Urol 2006; 16: 255-260.
Sahai A, Khan M S, Arya M, John J, Singh R, Patel H R H. The overactive bladder: review of current pharmacotherapy in adults. Part2: treatment options in cases refractory to anticholinergics. Expert Opinion Pharmacother 2006; 7: 529-538.
Sahai A, Khan M S, Arya M, John J, Singh R, Patel H R H. The overactive bladder: review of current pharmacotherapy in adults. Part 1: Pathophysiology and anticholinergics therapy. Expert Opinion Pharmacother 2006; 7: 509-527.
Sciarra F, Toscano V. Role of estrogens in human benign prostatic hyperplasia. Arch Androl 2000; 44:213-20.
Soderling S H, Beavo J A, Regulation of cAMP and cGMP signaling: new phosphodiesterases and new functions. Curr Opin Cell Biol. 2000; 12:174-179.
Spina D, Phosphodiesterase-4 Inhibitors in the treatment of inflammatory lung disease, Drugs 2003; 63:2575-2594
Truss M. C, Stief C, Machtens S, Wagner T, Jonas U (Eds) Pharmakotherapie in der Urologie, Springer 2001
Uckert S, Kuthe A, Jonas U, Stief C G, Characterization and functional relevance of cyclic nucleotide phosphodiesterase isoenzymes of the human prostate. J Urol 2001; 166:2484-2490.
Ying J, Yao D, Jiang Y, Ren X, Xu M, The positive effect of sildenafil on LUTS from BPH while treating ED Zhonghua Nan Ke Xue 2004 10:681-683.

1. A pharmaceutical composition for the treatment of a disease comprised in a group of diseases consisting of Benign Prostate Syndrome (BPS), Benign Prostate Hyperplasia (BPH), Benign Prostate Enlargement (BPE), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI) and pelvic pain, said pharmaceutical composition comprising a therapeutic agent which regulates the activity of soluble guanylate cyclase.

2. The pharmaceutical composition of claim 1, wherein the therapeutic agent which regulates the activity of soluble guanylate cyclase is a Guanylate-cyclase (sGC) stimulators and/or a sGC activator.

3. The pharmaceutical composition of claim 1, wherein the therapeutic agent which regulates the activity of soluble guanylate cyclase is a compound selected from the group consisting of 2-[1-(2-fluorobenzyl)-1H-pyrazolo[3,4-b]pyridin-3-yl]-5-(4-morpholinyl)-4,6-pyrimidinediamine (1), 2-[1-(2-fluorobenzyl)-1H-pyrazolo[3,4-b]pyridin-3-yl]-5-(4-pyridinyl)-4-pyrimidinamine (2), methyl-4,6-diamino-2-[1-(2-fluorobenzyl)-1H-pyrazolo[3,4-b]pyridin-3-yl]-5-pyrimidinyl(methyl)carbamate (3), methyl-4,6-diamino-2-[1-(2-fluorobenzyl)-1H-pyrazolo[3,4-b]pyridin-3-yl]-5-pyrimidinylcarbamate (4) and/or 4-({(4-carboxybutyl)[2-(2-{[4-(2-phenylethyl)benzyl]oxy}phenyl)ethyl]amino}methyl)benzoic acid (5).

4. The pharmaceutical composition of claim 1 further comprising at least one PDE5 inhibitor.

5. The pharmaceutical composition according to claim 4, wherein the PDE-5 inhibitor is selected from the group consisting of Vardenafil, Sildenafil, Tadalafil, Udenafil, Dasantafil, Avanafil, SLx2101 and LAS34179 or a salt, a hydrat or a hydrat of a salt thereof.

6. (canceled)

7. (canceled)

8. A pharmaceutical composition containing methyl-4,6-diamino-2-[1-(2-fluorobenzyl)-1H-pyrazolo[3,4-b]pyridin-3-yl]-5-pyrimidinylcarbamate (4) and/or 4-({(4-carboxybutyl)[2-(2-{[4-(2-phenylethyl)benzyl]oxy}phenyl)ethyl]amino}methyl)benzoic acid (5), for the treatment of neurogenic bladder, overactive bladder and interstitial cystitis.

9. A pharmaceutical composition containing at least methyl-4,6-diamino-2-[1-(2-fluorobenzyl)-1H-pyrazolo[3,4-b]pyridin-3-yl]-5-pyrimidinylcarbamate (4) and/or 4-({(4-carboxybutyl)[2-(2-{[4-(2-phenylethyl)benzyl]oxy}phenyl)ethyl]amino}methyl)benzoic acid (5) and at least Vardenafil, Sildenafil, Tadalafil, Udenafil, Dasantafil, Avanafil, SLx2101 and LAS34179 or a salt, a hydrat or a hydrat of a salt thereof for the treatment of neurogenic bladder, overactive bladder and interstitial cystitis.
